(19) United States
(12) Patent Application Publication
(10) Pub. No.: US 2007/0248655 A1
(43) Pub. Date: Oct. 25, 2007

(54) Lenticular shaped protective mouth sore discs

(76) Inventor: Jeffrey T. Haley, Mercer Island, WA (US)

Correspondence Address: GRAYBEAL, JACKSON, HALEY LLP, 155 - 108TH AVENUE NE, SUITE 350, BELLEVUE, WA, 98004-5973, US

(21) Appl. No.: 11/409,367

(22) Filed: Apr. 21, 2006

(51) Int. Cl. A61F 13/02

(52) U.S. Cl. 424/448; 514/54

(57) ABSTRACT

An adherent, soluble oral patch for delivering topical medication in the mouth having a lenticular shape. The structure of the oral patch is formed with a material that remains solid at human mouth temperatures and slowly dissolves or erodes in saliva, such as a network of long molecules such as starch, including corn starch. In some embodiments, the network is hydrophilic.

This application claims priority from U.S. Ser. No. 10/287,843 filed Nov. 5, 2002, U.S. Ser. No. 10/236,289 filed Sep. 4, 2002, and U.S. 60/344,577 filed Dec. 28, 2001.

### BACKGROUND

For treatment of health problems in the mouth or throat, people have for centuries held in their mouths a composition containing herbal or other medication for topical application. The oldest name for such a composition, derived from Latin and previously from Greek, is “troche”. A modern form of troche is the cough drop, so named because it was formed by “dropping” hot, viscous, sugar-based candy onto a sheet or into a mold where it cools to form the troche. Another modern form of troche is the throat “lozenge”, so named because it was in the shape of a diamond (like on playing cards), which is the meaning of the word “lozenge”. The structural characteristics of these types of troches are determined by their primary structural ingredients which are typically corn syrup or sugars, including sugar alcohols. The troches are relatively hard and are often irritating to tender surfaces such as canker sores. These troches are only mildly adherent to teeth and not significantly adherent to gums, cheeks, or lips.

To protectively cover a particular spot in the mouth, adherent oral patches have been developed. An oral patch typically includes one or more layers that do not dissolve entirely such as invented by Anthony et al. and disclosed. in U.S. Pat. No. 5,713,852. Another example of an oral patch is the DentiPatch which has one or more non-soluble thermo-plastic layers and lidocaine, offered for sale by Noven Pharmaceuticals, Inc. As used herein, the word “patch” does not include preparations that move about the mouth rather than resting in one place, such as cough drops, throat lozenges, or other troches, and therefore do not adhere and protect a spot. Nor does it include preparations that do not hold together as a single item when held in the mouth such as preparations of powder, liquid, paste, viscous liquid gel, or a tablet or troche that crumbles into a powder or paste when chewed or placed in saliva.

There are many uses for preparations containing a medication to be delivered topically in the mouth. In many treatment situations, it is advantageous to retain the preparation at one location in the mouth rather than allowing it to move in the mouth such as when talking. U.S. Pat. No. 6,139,861 issued to Mark Friedman surveys the known methods for adhering a slowly dissolving medication to a location within the mouth. These methods include two forms of adherent soluble patches, referred to by Friedman as “mucoadhesive erodible tablets”. These tablets are formed using polymers carboxymethylcellulose, hydroxymethylcellulose, polyacrylic acid, and carbopol-934.

For treatment of canker sores (aphthous ulcers) in the mouth, is better to keep a medication in the mouth as long as possible to obtain a topical application to the ulcer and its surrounding tissues. Existing tablets and lozenges are poorly designed for this purpose.

### SUMMARY

In one aspect, the invention is a candy-like blob to be retained in the mouth for at least one to four hours at a time. The blob can be formed in the shape of a tablet or a lozenge or a wafer or any other desired shape. The preferred shape is a thin lentil shaped disc.

To cause the blob to dissolve very slowly in saliva, a binder that dissolves slowly in saliva is incorporated. Binders that have been tested and found to work well include carrageenan, xanthan gum combined with kanjac gum, and agar. Another useable gum is gum arabic. Other gums similar to those listed, such as startches, also work.

In addition to causing the blob to dissolve very slowly in the mouth, the binder also moderates any strong flavor by spreading out over a long period of time the release of that flavor. Consequently, sweeteners and other products to mask the strong flavor are not required, although some users prefer a small amount of sweetener and some also prefer the addition of anise or other flavors.

In another aspect, the invention is an adherent oral patch comprising a molecular network formed as a unitary solid structure that remains a solid at human mouth temperatures. The network is preferably hydrophilic so that, even when applied to a wet mucosal surface in the mouth, it will tend to adhere by absorbing moisture from the mucosal surface. Preferably, the network slowly dissolves in saliva so that the patch merely dissipates over time and the patch never has to be removed from the mouth.

The adherent oral patch will adhere to teeth, gums, cheek, lips, or tongue without the user first drying saliva from the tissue. If the patient merely places the oral patch in his or her mouth and holds it in the desired location for 10 to 40 seconds, it will then adhere to the tissues that it has been touching without movement, even though those tissues are wet. This is far easier for patients to use than requiring that the tissue first be dried with a towel before the adherent oral patch is placed. If the patient wants to use an oral patch in the lip or under the tongue, the oral patch can easily be removed for talking and then easily be replaced without using a towel or a mirror.

A desired medication is located within the network. The network may be comprised of a thermo gel having a melting temperature higher than human mouth temperatures. Alternatively, the network may be comprised of a complex carbohydrate, such as cellulose, pectin, maltodextrin, or starch from potato, rice, corn, or wheat. Also, the network may be comprised of a hydrogel with a melting temperature higher than temperatures in the human mouth formed of amino acids, such as peptides.

### BRIEF DESCRIPTION OF THE DRAWINGS

FIG. 1 shows a side view of an adherent, soluble oral patch.

FIG. 2 shows a top view of the same oral patch.

### DETAILED DESCRIPTION

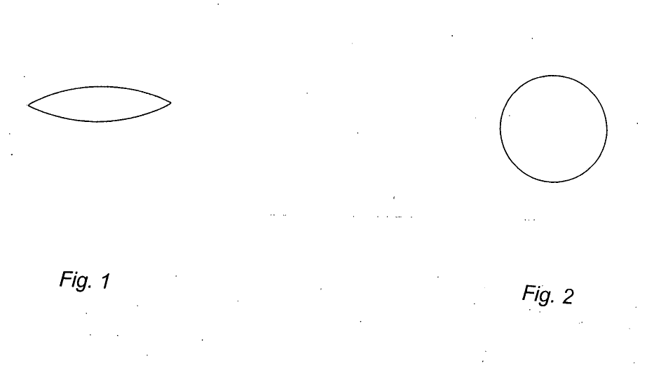

FIG. 1 shows a preferred shape for the oral patch. It is made with slowly dissolving hydrocolloids so that that it typically lasts in the mouth for at least one to six hours. The patch can be formed in the shape of a tablet or a lozenge or a wafer or any other desired shape. A preferred shape is a thin lentil shaped disc as shown in FIG. 1.

A requirement for the patch is that it remains a solid, rather than melting, at human mouth temperatures. So that the oral patch will slowly erode, it should be made of a material with a low to moderate rate of disintegration in warm saliva. If the patch does not erode fast enough, water soluble medication will be drawn out of the network faster than the network erodes.

Many different compositions can be used to form the patch. For ease of manufacturing, it is convenient if the patch is comprised of a thermo gel having a melting temperature higher than human mouth temperatures. This allows the entire mixture to be a liquid at temperatures far above human mouth temperatures and allows the patch to be formed by cooling the mixture. Readily available materials that form such a gel include agar, in various forms, carrageenan, in most of its forms, particularly kappa carrageenan, konjac gum, locust bean gum, and xanthan gum. All of these materials form a thermo gel that is sufficiently elastic or plastic or a combination thereof for the network to feel soft in the human mouth if it is adequately hydrated.

Synthetic hydrogels may be used. Protein-based hydrogels are usually prepared using proteins extracted from natural sources, but they may be synthesized, such as with diblock copolypeptide amphiphiles, as taught by Nowak, et. al, “Rapidly Recovering Hydrogel Scaffolds From Self-Assembling Diblock Copolypeptide Amphiphiles”. Nowak, A. P.; Breedveld, V.; Pakstis, L.; Ozbas, B.; Pine, D. J.; Pochan, D.; Deming, T. J. Nature, 2002, 417, 424-428. The use of synthetic materials allows adjustment of copolymer chain length and composition. Synthetic hydrogels may also be made from polysaccharides and synthetic block copolymers which form thermoreversible gels and allow the solubilisation of hydrophobic medications for controlled release, as taught by Williams, P A, at the Centre for Water Soluble Polymers, North East Wales Institute, Plas Coch, Mold Road, Wrexham, Wales.

Instead of forming the patch with a true hydrogel, the patch may be formed with a complex carbohydrate, such as cellulose, pectin, starch from corn, maltodextrin or other polysaccharides. Forming of hydrated network structures out of such materials is well known in the candy making industry for making gummy candies. Or the patch may be formed with a combination of a true hydrogel and a complex carbohydrate.

The adherent oral patch is suitable for use with all of the medications mentioned in U.S. Pat. No. 6,139,861 issued to Friedman, including anesthetics, such as benzocaine, steroids, such as a glucocorticoid steroids, and non-steroidal anti-inflammatory drugs such as naproxen sodium, ibuprofen, acetaminophen, and ketoprofen. The medication may also be an antimicobial, such as an anti-fungal for treatment of candida organisms (thrush), such as nystatin, clotrimazole, miconazole, or fluconazole. The medication may be intended for treatment of canker sores (aphthous ulcers), including pharmaceutical antibiotics such as tetracycline, penicillin, or amoxicillin, or other canker sore treatment medications such as licorice root extract or amlexanox.

If the patch is formed of a hydrogel as described above, it may be manufactured by processes well known in the candy making industry. The process is to form a well-hydrated mixture at temperatures just below the boiling temperature of water so that water does not boil off and yet the hydrogels are fully activated for gelling when the product is cooled. In this process, the network can be formed of a combination of a true hydrogel such as xanthan gum with locust bean gum or with konjac gum and a complex carbohydrate such as cellulose or pectin or starch such as corn starch.

The mixture is poured or squirted into molds. Closed molds may be used such as in an injection-molding machine. Because the mixture typically requires about 2 hours to form a strong enough oral patch for de-molding, it is preferable to intermittently move trays of two-part molds, upper and lower, under pump depositer injector nozzles. The nozzles fit into holes in the upper mold located at the center of each oral patch. After de-molding, the upper molds are used again for another batch. The lower molds may be plastic lined, in which case the plastic becomes a part of the final packaging. A suitable size for each oral patch is 0.8 grams poured into the mold.

If the oral patches are deposited in molds formed in a tray, the tray is stored in a drying room until the oral patches lose a suitable amount of moisture. A suitable method of drying in trays is to expose them without convection to room temperature and humidity for 3 days or, with convection, for 24 hours. In the drying process, the oral patches lose about 47% of their weight, so an oral patch that started at 0.8 grams poured into the mold becomes 0.42 grams. The trays are then sealed with a film or foil lid that is adhered by conventional heat-sealing techniques.

For most applications, most users prefer that the oral patches be medium dry to dry. With this starting dryness, the oral patches are more adherent and have more integrity so they can be removed for talking or eating and then replaced.

While particular embodiments of the invention have been described above, the scope of the invention should not be limited by the above descriptions but rather limited only by the following claims.

1. A lenticular shaped mucoadhesive disc for treatment of mouth sores containing one or more binder ingredients that adheres to mucosa and slowly dissolves in saliva when held in a human mouth on a mouth sore.

2. The disc of claim 1 wherein the binder ingredient is selected from a group comprising starch, konjac gum, locust bean gum, and xanthan gum.

3. The disc of claim 2 wherein the binder ingredient is starch from corn.

4. The disc of claim 1 wherein, when placed in an average human mouth and not chewed, the disc does not disintegrate into more than one piece.

5. The disc of claim 1 wherein, when placed in an average human mouth and not chewed, the disc lasts for more than one hour before completely dissolving.

6. The disc of claim 1 further comprising an anesthetic selected from a group comprising benzocaine and lidocaine.

7. A method of treating lesions in a human mouth by holding in the mouth on the sore a mucoadhesive disc containing one or more binder ingredients that adheres to mucosa and slowly dissolves in saliva when held in the mouth on a mouth sore.

8. The method of claim 7 wherein the binder ingredient is selected from a group comprising starch, konjac gum, locust bean gum, and xanthan gum.

9. The method of claim 8 wherein the binder ingredient is starch from corn.

10. The method of claim 7 wherein, when placed in an average human mouth and not chewed, the disc does not disintegrate into more than one piece.

11. The method of claim 7 wherein, when placed in an average human mouth and not chewed, the disc lasts for more than one hour before dissolving.
